(19) United States
(12) Patent Application Publication
(10) Pub. No.: US 2022/0130512 A1
(43) Pub. Date: Apr. 28, 2022

(54) GENERATING PERSONALIZED FOOD GUIDANCE USING PREDICTED HUNGER

(71) Applicant: Zoe Limited, London (GB)

(72) Inventors: Patrick James Wyatt, London (GB); Jonathan Thomas Wolf, London (GB)

(73) Assignee: Zoe Global Limited

(21) Appl. No.: 17/080,403

(22) Filed: Oct. 26, 2020

(51) Int. Cl. G16H 20/60; G16H 10/60; G16H 50/20; G16H 50/30; G16H 50/50; A61B 5/00; G09B 19/00

(52) U.S. Cl. CPC G16H 20/60; G16H 10/60; G16H 50/20; G16H 50/30; G16H 40/67; A61B 5/4866; A61B 5/486; G09B 19/0092; G16H 50/50

(57) ABSTRACT

Techniques are disclosed herein for generating personalized food guidance using predicted food hunger. Using the technologies described herein, instead of providing food guidance that is generalized for a group of individuals, personalized food guidance is provided that takes into account an individual's personalized responses to foods, including the predicted hunger of an individual. A nutritional service generates a hunger score that predicts a hunger level of an individual at a time (or for more than one time) after the individual has or is planning to consume food. The nutritional service uses the hunger score to generate the food guidance. Providing an individual with personalized food guidance can make choosing food easier and healthier.

### BACKGROUND

Obesity represents a major global health challenge with its prevalence in adults and children increasing worldwide. Currently, effective interventions to prevent or reverse obesity have, for the most part, failed to be translated into successful personalized strategies that are likely to produce sustained healthy behavior. A number of different factors make intentional weight loss difficult and facilitate weight regain after weight loss. In addition to decreases in resting energy expenditure, increases in appetite while dieting can make it difficult for an individual to lose weight.

Further, providing guidance to individuals about what to eat and how to change a diet can be very difficult and confusing. Not only do individuals have a large variety of food choices, but food that is healthy for one individual may not be healthy for another individual. Age, sex, weight, the microbiome of an individual, as well as other factors affect what foods an individual should select to eat. For example, while low carbohydrate food or low-fat food may be beneficial for one individual, that same low carbohydrate or low-fat food choice may not be beneficial for another individual.

### BRIEF DESCRIPTION OF THE DRAWINGS

FIG. 1 is a block diagram depicting an illustrative operating environment in which personalized food guidance is generated using predicted hunger of an individual.

FIG. 2 is a block diagram depicting an illustrative operating environment in which personalized food guidance is generated using predicted biomarkers and predicted hunger of an individual.

FIG. 3 shows predicting hunger using a hunger prediction service/scoring service.

FIG. 4 illustrates a relationship between reported hunger levels and glucose levels.

FIG. 5 illustrates differences in calories consumed after eating different meals.

FIG. 6 illustrates increased hunger and calorie consumption based on glucose drops.

FIG. 7 is a flow diagram showing routine illustrating aspects of a mechanism disclosed herein for generating food guidance that is personalized for an individual using predicted hunger of an individual.

FIG. 8 is a flow diagram showing routine illustrating aspects of a mechanism disclosed herein for generating hunger scores that are personalized for a user.

FIG. 9 is a block diagram depicting an illustrative operating environment in which a data ingestion service receives, and processes data associated with data associated with at home measurements relating to intake and/or responses to food.

FIG. 10 is a computer architecture diagram showing one illustrative computer hardware architecture for implementing a computing device that might be utilized to implement aspects of the various examples presented herein.

### DETAILED DESCRIPTION

The following detailed description is directed to technologies for generating personalized food guidance using predicted hunger. As briefly discussed above, personalized strategies that produce sustained healthy behavior relating to eating have, for the most part, failed to be successful. Using technologies described herein, instead of providing food guidance that is generalized for a group of individuals, a nutritional service considers a predicted hunger of an individual and the individual's predicted responses to food when generating food guidance. Providing an individual with personalized food guidance that considers a predicted hunger of the individual can make choosing food easier and healthier, allow them to eat less as they may not remain hungry after eating, and help them to understand how to improve their existing diet.

In some configurations, the nutritional service may generate a hunger score that predicts a hunger level of an individual at a time (or for more than one time) after the individual has or is planning to consume food. According to some examples, the nutritional service uses the individual's glucose responses and/or other individuals glucose responses, and other data, to generate the food guidance. For instance, the nutritional service may use glucose responses, fat responses, microbiome data, data about the individual's overall health, potential health risks for the individual, and/or other data associated with the individual to generate the food guidance.

Traditionally, glucose response has been measured in the first two hours after a meal to calculate a glycemic response (e.g., used by clinicians to diagnose diabetes or nutritionists to measure Glycemic Index). Research has also focused on the part of the glycemic response that rises above the initial (baseline) glucose value, which may be referred to herein as “glycemic rise”. Using technologies described herein, however, glycemic responses can be measured and/or predicted much farther out in time than two hours from eating a particular food/meal. For example, the nutritional service may generate a hunger score for an individual based on a predicted glucose response that is more than two hours past a meal.

Further, instead of relying on the glycemic rise, the portion of the glycemic response that drops below the baseline glucose level for a user (which may be referred to herein as “glucose drop”) may be detected and/or predicted. The “baseline glucose level”, which may also be referred to herein as “baseline level” refers to a median pre-meal glucose level determined from a group of users that was recorded as a response to different meals eaten. As will be discussed in more detail below, a glucose drop can indicate hunger for a user, and when a user becomes hungry, the user typically will consume more calories. In some configurations, the nutritional service predicts the glucose drop for an individual (e.g., 2-4 hours after consuming food), and uses the predicted glucose drop to generate the food guidance.

According to some examples, the personalized food guidance generated by the nutritional service may include guidance data such as but not limited to personalized health scores (e.g., scores related to hunger, glucose, fat, . . . ), guidance/recommendations on food that has not yet been eaten, and/or other guidance data (e.g., via a User Interface (UI)) on food/meal(s) that have already been eaten). The guidance data may be used by an individual to help them understand how they can improve their diet.

In some configurations, the nutritional service may generate food guidance such that perceived hunger by the individual is lessened after eating recommended foods. According to some examples, the nutritional service can select food for the individual to eat to minimize glucose drops. In some configurations, the nutritional service predicts the glucose drop for an individual (e.g., 2-4 hours after consuming food), and uses the predicted glucose drop to determine food to eat that attempts to reduce/eliminate the hunger of the individual. The glucose responses can be in part predicted by the nutritional service based on various factors, such as the amount of sugar in a meal and personal factors such as insulin sensitivity. In some examples, the glucose drop of the glucose response for an individual may be used by the nutritional service to predict both perceived hunger and the number of calories consumed at some point in time (e.g., 3 or more hours) after the meal is consumed. This may be linked to a person's insulin sensitivity and the sugar content, but not necessarily the glycemic index, of the meal.

In other examples, the nutritional service can identify foods which minimize energy dips for an individual. In further examples, the nutritional service can identify which foods minimize snacking a few hours after consuming food, such that if those foods are consumed by an individual, cause less hunger at 3 or 4 hours. As a result, if the individual follows the food guidance provided by the nutritional service, the individual may not snack as much and/or may choose to eat smaller calorie meals, which over time will lead to reduced weight gain or weight loss.

The nutritional service may also use other data when generating food guidance. For instance, the nutritional service may use properties of the food that can be measured independently of the user, such as grams of fiber, level of food processing, type of fiber, glycemic index, sugar content, salt content, alcohol content, whether the food is fermented, whether the food is a high polyphenol food such as dark chocolate or vegetables, whether the food is meat, the composition and source of fat in the food, the composition of the fiber in the food, the production method, preparation methods applied by the user (such as cooking method), the inclusion of particular ingredients such as (but not limited to) flavor enhancers or emulsifiers, the method by which the food was preserved, the location where the food was produced, the method by which animals contributing to the product were reared, caught or slaughtered, and the like. The nutritional service may weight these properties differently depending upon the individual (for example people with high lipid responses may be guided more strongly against alcohol and in favor of fiber) In some configurations, the nutritional service may generate different scores that are combined to generate a single overall rating, which may be referred to as a “meal score” of the food in a particular context.

As such, using the techniques described herein, rather than a user looking at food data, such as a glycemic index associated with a particular food, or guessing whether or not a food is good for them, the nutritional service may generate food guidance that considers the predicted hunger of a user if a recommended meal is eaten. By adjusting a food/meal score based on a predicted hunger score for the food, may make it easier for an individual to choose the right food at a particular moment. In addition, a personalized meal score reflecting an individual's personal responses may be more valuable than generic food label data that does not change if an individual has a risk of diabetes or cardiovascular disease.

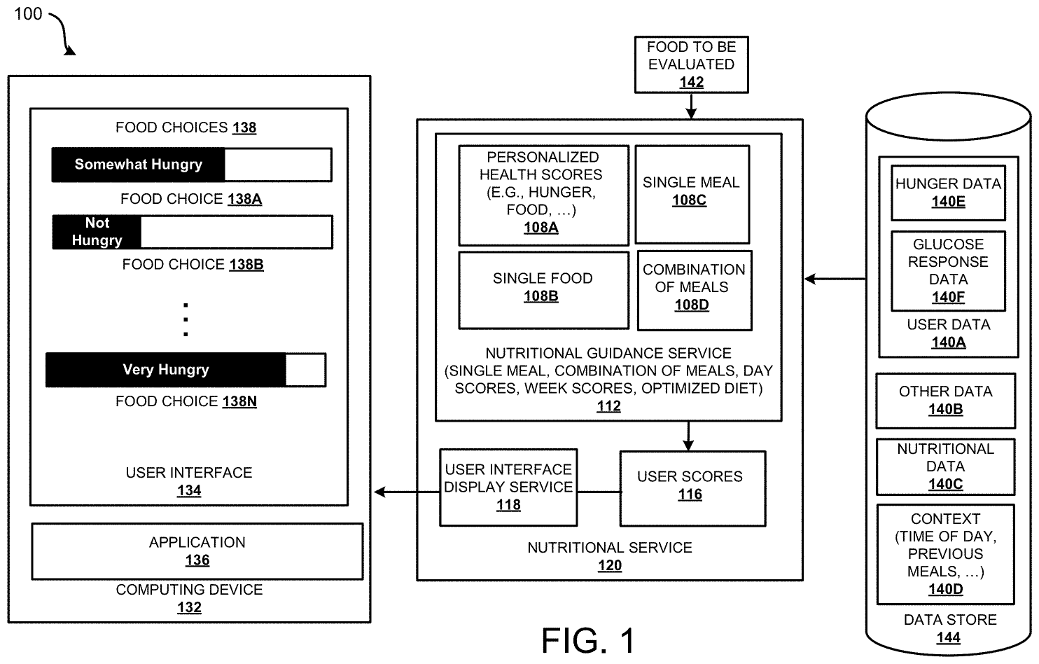

Using the food guidance and viewing the UI elements presented to the user that indicate the different generated scores makes it easy for a user to determine what foods to eat as well as how well the user has eaten. In some examples, the nutritional service may also use food/meal scores to rank a list of foods or to generate a suggested diet plan which consists of a set of foods chosen to be optimal for the individual when combined. In some cases, these foods will also incorporate the individual's food preferences such as being vegan or not liking tomatoes. More details relating to generating personalized food recommendations are described below with reference to FIGS. 1-10.

FIG. 1 is a block diagram depicting an illustrative operating environment 100 in which personalized food guidance is generated using predicted hunger. As illustrated, environment 100 includes a nutritional service 120, a nutritional guidance service 112, a UI display service 118, a data store 144, and a computing device 132.

As shown in FIG. 1, the operating environment 100 includes one or more computing devices 132 in communication with a nutritional service 120. In some examples, the environment 100 may be associated with and/or implemented by resources provided by a service provider network such as provided by a cloud computing company. The environment 100 may include a collection of computing resources (e.g., computing devices such as servers). The computing resources may include a number of computing, networking and storage devices in communication with one another. In some examples, the computing resources may correspond to physical computing devices and/or virtual computing devices implemented by one or more physical computing devices.

The data store 144 may include a variety of data 140. In some configurations, an individual may generate and provide data 140 using a variety of at home biological collection devices, which collect a biological sample which requires a biological assay to be performed to generate electronic data 140. Some of the data 140 may be biomarker data, such as blood glucose response data 140F collected by a Continuous Glucose Monitoring (CGM) device, as well as other types of data, such as hunger data 140E that is a perceived hunger of an individual at some point in time (e.g., before/after a meal). Some of this data may be non-biomarker data such as photos, time stamps, as well as other data associated with a user or group of users.

According to some examples, digital devices, such as a CGMs and applications running on digital devices (e.g., mobile phones) may be used by individuals to provide data, such as the glucose response data 140F and the hunger data 140E. For example, the glucose monitor may provide glucose response data 140F, when authorized by an individual, to the nutritional service 120, and the individual may input hunger data 140E via a user interface that indicates a hunger level (e.g., 1-10, 1-100, or some other indicator of perceived hunger) of the individual and/or other data.

In some configurations, the nutritional service 120 uses data obtained from many individuals (e.g., users that participated in a study) that includes glucose response data and appetite/hunger data associated with the large number of individuals' responses to standardized meals and measured responses to the standardized meals and non-standardized meals. As used herein a standardized meal is a meal in which an individual eats foods/portions that are specified. A non-standardized meal, which may be referred to herein as “free-living meals”, is a meal in which an individual chooses the foods/portions to eat.

In some configurations, an individual is presented with UI elements, such as UI elements 138, that present food guidance. The UI elements may be used to represent foods to eat, scores, such as a hunger score associated with a food choice (e.g., food choice 138A-138N), a food score, a meal(s) score, and the like. In some examples the meal score reflects the overall predicted effect that this meal will have on the individual's metabolism and provides a means for individuals to compare different meals.

When comparing two foods/meals, it is often not immediately obvious which food is better, as one food may lead to a better response in one biomarker (for example blood sugar), while another may lead to a better response in another biomarker (e.g. blood fat). In some configurations the nutritional service 120 may calculate the relative importance of the different responses for that individual and show a simple score and/or some other indicator to allow an individual to easily compare any two meals or understand how they could improve their diet.

The nutritional service 120 may combine multiple data sources about the individual's response to each food in such a way that the nutritional service considers the individual's own personalized risks, so as to optimize the generated food guidance for each individual based upon their own body's responses. In some examples, the nutritional service 120 may use one or more other services, to score the individual's responses to food on more than one dimension (e.g., the impact of the food on biomarkers such as glucose, triglycerides, insulin, energy and its impact on altering hunger of an individual, shifting the microbiome towards a specific goal, and the like). In some cases, these scores may depend on factors that change rapidly such as the time of day, what was eaten previously or the amount of sleep.

Some of this data 140 may be nutritional data 140C associated with individual foods. In some configurations this may include data from nutritional databases such as fiber or grams of sugar. Some of this data may come from individuals who have recorded eating such food and shared their own biological responses to this food such as blood glucose response data 140F and/or reported hunger data 140E. By aggregating this data and using other information about these individuals, in some configurations some of the nutritional data for this food such as its impact on blood sugar can be calculated.

An individual may also provide data 140 using computing device 132. In some configurations an individual can input data 140 into one or more software applications 136. For example, an individual may enter the food they consumed, an indication of how hungry they feel at different times after/before consuming food, a value indicated by a measurement device, their waist measurement, and the like. Alternatively, and/or in addition to the above, data 140 generated by other measurements can be used to assist in determining when a food was eaten, and/or a test was performed. For example, in some cases a CGM can be used to confirm the start point of a meal and record data after the meal.

The nutritional service 120 can access the data 140 when generating the personalized nutritional guidance and/or scores for a particular user. In some examples, the nutritional service 120 utilizes both data associated with a particular individual for which the food guidance is generated for and data from other individuals. In other examples, the data utilized is associated only with the user. According to some examples, the data 140 can additionally include data obtained from a clinical setting, which is typically more accurate than at home measurements.

In some examples, the nutritional service 120 may utilize one or more machine learning mechanisms. For example, the nutritional service 120 can use a classifier to classify the data within a classification category. In other examples, the nutritional service 120 may generate one or more scores using one or more machine learning mechanisms.

As briefly discussed above, the nutritional service 120 generates personalized food guidance using predicted hunger and food responses of an individual. Instead of providing a user, such as a user of computing device 132 with food guidance that is generalized for a group of individuals, the nutritional service 120 generates personalized food guidance that takes into account the user's responses to foods, such as the individual's glucose and fat responses, the individual's microbiome, the individual's overall health, potential health risks for the individual, the predicted hunger of the individual, and/or other user data 140A that is associated with the individual.

In some configurations, the nutritional service 120 can generate a hunger score that predicts a hunger level of an individual at a time (or for more than one time) after the individual has or is planning to consume food. According to some examples, the nutritional service 120 predicts the individual's glucose responses to one or more foods using the individual's glucose response data 140F and/or other individuals glucose response data, and other data, to generate the food guidance. For instance, the nutritional service may use glucose responses, fat responses, microbiome data, data about the individual's overall health, potential health risks for the individual, and/or other data associated with the individual to generate the food guidance.

Instead of relying on the glycemic rise, the nutritional service predicts the glucose drop for an individual based on one or more food choices. According to some configurations, the nutritional service 120 uses a nutritional guidance service 112 to generate a variety of personalized food guidance data that may be provided to a user via UI 134, or through some other mechanism. The personalized food guidance data may include data such as but not limited to food choices 138 that indicate how particular food choices affect a perceived hunger of an individual. The nutritional guidance service 112 may also generate personalized health scores (e.g., hunger, glucose, fat, . . . ) 108A, guidance/recommendations/scores on single food items (already eaten or not yet eaten) 108B, single meals (already eaten or not yet eaten) 108C, combination of meals (already eaten or not yet eaten) 108D, and/or other guidance data (not shown).

In some configurations, the nutritional service 120 may generate food guidance such that perceived hunger by the individual is lessened after eating recommended foods. According to some examples, the nutritional service 120 can select food for the individual to eat to minimize glucose drops. In some configurations, the nutritional service 120 predicts the glucose drop for an individual (e.g., 2-4 hours after consuming food), and uses the predicted glucose drop to determine food to eat. The glucose responses can be in part predicted by the nutritional service based on various factors, such as the amount of sugar in a meal and personal factors such as insulin sensitivity. In some examples, the glucose drop of the glucose response for an individual may be used by the nutritional service to predict both perceived hunger and the number of calories consumed at some point in time (e.g., 3 or more hours) after the meal is consumed. This may be linked to a person's insulin sensitivity and the sugar content, but not necessarily the glycemic index, of the meal.

In other examples, the nutritional service 120 can identify foods which minimize energy dips for an individual. In further examples, the nutritional service 120 can identify which foods minimize snacking a few hours after consuming food, such that if those foods are consumed by an individual, cause less hunger at 3 or 4 hours. As a result, if the individual follows the food guidance provided by the nutritional service, the individual may not snack as much and/or may choose to eat smaller calorie meals, which over time will lead to reduced weight gain or weight loss.

In some examples, the nutritional guidance service 112 provides the guidance data to a user interface display service 118 to provide to the user via UI 134. The guidance data displayed in UI 134 may be used by an individual to help them understand how they can improve their diet. For instance, a user may receive guidance data by viewing UI 134 on a mobile device, or some other device, to readily determine how good/bad a particular food/meal is for them.

In the current example illustrated in FIG. 1, an individual may readily determine what food choice 138 will make them feel less hungry after eating the meal. In the current example, the food choice 138A may make the user feel “somewhat hungry”, the food choice 138B may make the user feel “not hungry”, and the food choice 138N may make the user feel “very hungry”. In some configurations, the hunger score may be presented differently (e.g., a numerical score) and/or with different colors. For example, a “green” UI element may indicate that the food choice 138B does not cause the user to be hungry, a “yellow” UI element may indicate that the food choice 138A may cause the user to be somewhat hungry, and a “red” UI element to indicate that the food choice 138N to be “very hungry”. As will be discussed in more detail below, the foods eaten earlier by an individual may impact the scores, including the hunger score, for food eaten later in the day. Stated another way, the same meal eaten at different times may be scored differently, and cause other scores to be different, for the same user depending on what foods the user has eaten before that meal.

According to some examples, the user may utilize the UI 134 to record dietary intake (e.g., foods eaten), hunger levels, and may receive notifications and reminders to complete tasks at specific times, such as when their test lunch meals and DBS were due and asked to report their hunger and alertness levels on visual scales.

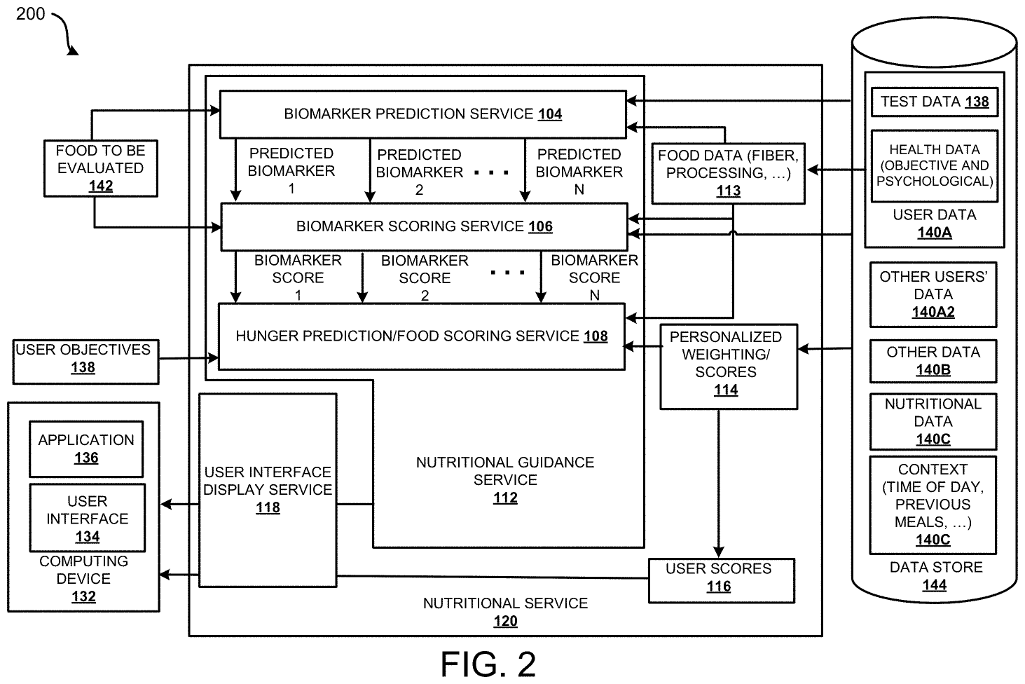

FIG. 2 is a block diagram depicting an illustrative operating environment 200 in which personalized food guidance is generated using predicted food responses and predicted hunger. As illustrated, environment 200 includes more details as compared to environment 100. For example, environment 200 includes biomarker prediction service 104, biomarker scoring service 106, hunger prediction/food scoring service 108 as part of the nutritional guidance service 112. In other configurations, the services may be located in different environments and/or locations.

In some examples, the nutritional guidance service 112 may receive a single input (e.g., the glucose rise linked to foods). In this example, the nutritional guidance service 112 generates a prediction of the biomarker of interest (e.g., the glucose drop) using biomarker prediction service 104 and a score for that single biomarker using the biomarker scoring service 106. However, in other examples where more than one aspect of the food is calculated, nutritional guidance service 112 predicts many different biomarkers (e.g., biomarkers 1-N) of the assessed aspects (glucose responses including glucose rise and glucose drop, blood fat response, and the like), scores each biomarker change individually using the biomarker scoring service 106 and weights the scores so that the scores are comparable and then combined into a single score using hunger prediction/food scoring service 108.

In some examples, in addition to utilizing biomarker inputs, the nutritional guidance service 112 also utilizes non-biomarker inputs such as but not limited to food data 113 such as fiber content, amount of processing on the food (e.g., high, medium, low), polyphenol content, fermentation and the like. In some examples the nutritional guidance service 112 uses information about the individual to determine personalizing weighting 114 for combining these scores, which determine how to combine the different biomarker scores 106 and food data, 113 into a single score.

The biomarker prediction service 104 generates a prediction of the postprandial response of the user's biomarker to the food being evaluated. The biomarker scoring service 106 generates scores that are personalized for a particular user. The scores generated by the biomarker scoring service 106 are related to the predicted health impact of these biomarker changes on the particular user, so for example a small rise in blood fat after a meal might have no negative impact on health and so the score for blood fat might be at the best level for that meal but a large rise in blood fat 6 hours after a meal might be detrimental to health and lead to a bad score for blood fat for that meal for that person. As another example, a predicted large glucose drop after a meal may lead to a high hunger score for an individual, whereas a predicted small glucose drop leads to a lower hunger score that indicates that the user will be less hungry as compared to the high hunger score.

The nutritional service 120 may then use these biomarker scores to generate food scores for one or more foods/combination of foods/meals to make individual food recommendations by generating personalized meal scores for any meal using food scoring service 108. In some examples, the hunger prediction/food scoring service 108 uses personalized weighting/scores 114 to determine how to weight the different biomarker scores when combining them to generate a single meal score. For example, the hunger prediction/food scoring service 108 might need to combine a glucose rise, a glucose drop, a hunger score, a blood score and a gut health score, and might do so by weighting each of these scores and then combining them.

In some examples, if the personalized impact of a food is to be calculated by using predictions of the hunger, glucose, and/or triglycerides levels after eating that food combined with the food's impact on the microbiome for that individual, generating a meal score by the hunger prediction/food scoring service 108 may consist of using the personalized weighting/scores to combine these scores. The nutritional service 120 is designed to do this such that it recommends the best possible food for the individual based upon the inputs provided. In other examples the nutritional service 120 will use information beyond biomarker predictions as inputs into its biomarker scoring, for example it may take into account family health history, measures of blood chemistry taken in a fasting state such as baseline glucose or triglycerides, or measures of blood chemistry that do not change postprandially such as cholesterol or HbA1c. The nutritional service 120 can also utilize one or more data analysts, machine learning, and/or some other mechanism to generate a quality score for the data.

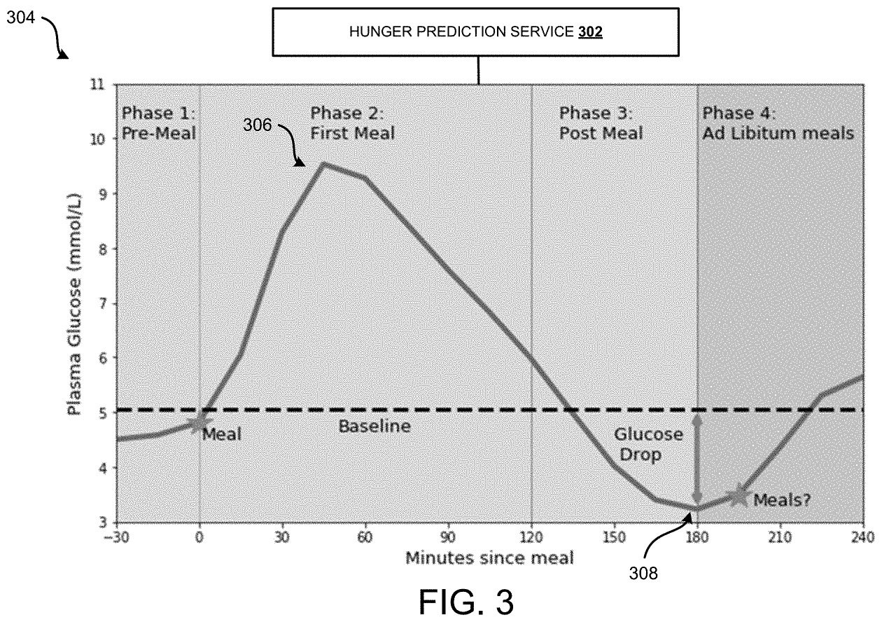

FIG. 3 shows predicting hunger for an individual using a hunger prediction service 302. As discussed above, a biomarker scoring service 106 scores predicted biomarkers obtained from biomarker prediction service 104. In some examples, the hunger prediction service 302 may generate data that predicts a glucose response of an individual in response to eating food such as illustrated by graph 304. As illustrated, the graph 304 includes a glucose response including a glucose rise 306 and a glucose drop 308 that is predicted across different phases that occur before eating food and after eating food. In the current example, four phases are shown that include a pre-meal phase 1, a first meal phase 2, a post meal phase 3, and an ad libitum meal(s) phase 4. While four phases are illustrated, more or less phases may be used. Further, the time period of each phase may be adjusted to include more/less time.

As shown, the pre-meal phase 1 includes the time period of up to 30 minutes before the meal starts. Pre-meal phase 1 is characterized by a pre-meal glucose level, which may be predicted using the nutritional service 120 and/or determined from a glucose measuring device. In some examples, the glucose level of the pre-meal phase 1 may be below the baseline glucose level determined from a group of users.

The first meal phase 2 includes the time from when food is eaten to two hours since eating. The first meal phase 2 includes the consumption of the total calories consumed in the 2-hour period. As can be seen, after eating the meal, the glucose level rises for a period and then begins to drop. The peak glucose level may be referred to herein as the “glucose Rise %”, which is the maximum level 306 above the baseline within the 2-hour period, as a percentage of the baseline level. “iAUC0-2 h” is the incremental area under the glucose curve, measured relative to the baseline level.

The post-meal phase 3 is the time period from +2 hours after eating the meal to +3 hours after the meal. As can be seen, the post-meal phase 3 includes the glucose drop where the predicted glucose level for the individual drops below the baseline level. As discussed, the larger the glucose drop 308, the hungrier an individual will typically feel. The glucose drop may be expressed as a score, a percentage, or some other way, in order to adjust for differences in participants' baseline levels. Positive glucose dip values indicate states of mild hypoglycemia, negative glucose dip values indicate that blood glucose levels remain elevated above baseline level.

As illustrated, the “glucose drop” is largest for this user at the three hour mark but may be largest (the difference between the baseline and the lowest glucose level) in hours 2-4 hours post meal. As discussed above, larger glucose drops lead to higher declared levels of hunger as indicated by hunger data collected via an application, such as application 136.

The ad libitum meals phase 4 is the time period after 180 minutes from eating the meal. In some examples, users may record the food they eat throughout the day and this data may be used to adjust the predictions for one or more of the users. As also discussed above, users may provide input after/before a meal as to how hungry they feel. This hunger data may also be used to adjust the predictions.

In some examples, the hunger prediction service 302 may combine postprandial and static elements to generate a score. This score generated by the hunger prediction service 302 is designed to reflect a hunger level for a particular user in response to eating one or more foods. In some examples the hunger prediction service 302 may generate a hunger score that is based on the glucose drop, and/or the glucose rise and/or other data. Generally, the higher the hunger score, the more likely an individual may consume more calories.

After generating the predicted glucose response and/or the hunger score, the hunger data may then be provided to a hunger prediction/food scoring service 108 as illustrated in FIGS. 1 and 2. This score may be weighted according to weights 114 associated with a particular individual to score the meal. In some examples, the biomarker score may aggregate a number of other biomarkers, for example there may be an “aggregate harmful lipid marker” which may be calculated by the biomarker prediction service/scoring service 302, or some other component, by combining a number of biomarkers that might be measured using a metabolite screen, at a particular time (fasting, or at a set time point after a challenge meal or meals).

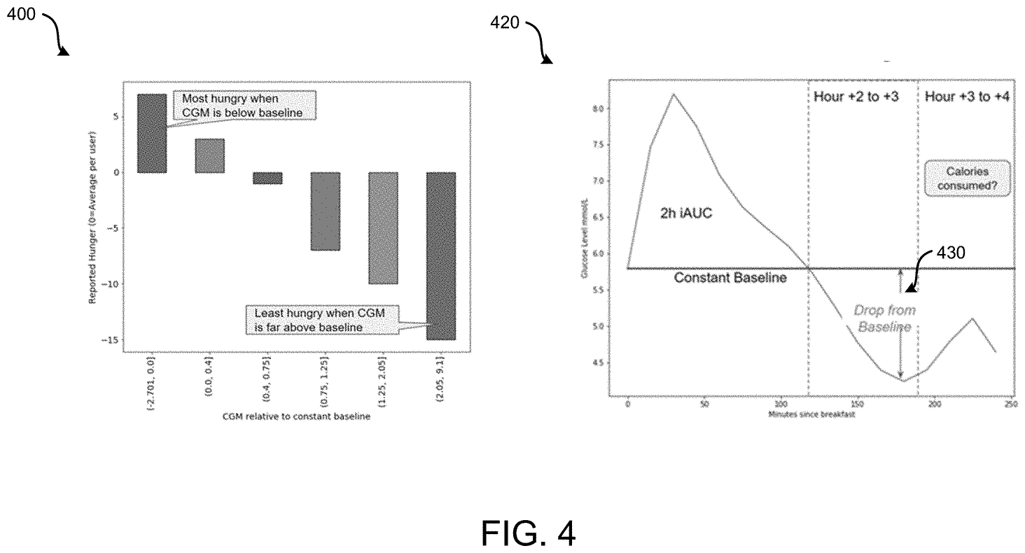

FIG. 4 shows graph 400 that illustrates a relationship between reported hunger levels and glucose levels and graph 420 that shows an exemplary blood glucose response curve. Referring to graph 420, it can be seen that as the glucose drop 430 is largest near the 3-hour mark. As discussed above, the nutritional service 120 can utilizes data obtained from a number of users to determine a baseline glucose level. The data may be glucose levels obtained at different times before/after eating a meal, and across a diverse set of meals (e.g., standardized meals and free-living meals). Similarly, hunger data may be utilized by the hunger prediction service to determine baseline hunger values. In some examples, the users record their hunger levels immediately before or after consuming the meal and log the calories that they consumed in the following 2 hours.

Referring to graph 400 it can be seen that when the glucose drop is the largest, the reported hunger by a user is highest. As the glucose level increases and the glucose drop is not as large, the hunger level of an individual decreases. Graph 420 shows the two-hour Incremental Area under the Curve (iAUC) where the curve is the blood glucose response curve. In some examples, the blood glucose response curve may be measured by a CGM or obtained using some other method or technique. As discussed above, perceived hunger as illustrated in graph 400 may be measured using a mobile application that presents a UI 134 that may ask users to rate their hunger levels on a scale from 1-100, at intervals before/after the consumption of meals.

As can be seen by referring to the graphs 400 and 420, if the glucose drop can be reduced/eliminated, the hunger of an individual can be reduced. As discussed above, the nutritional service 120 may generate food guidance that is directed at reducing the glucose drop of an individual such that the user feels less hungry and is less likely to eat more calories.

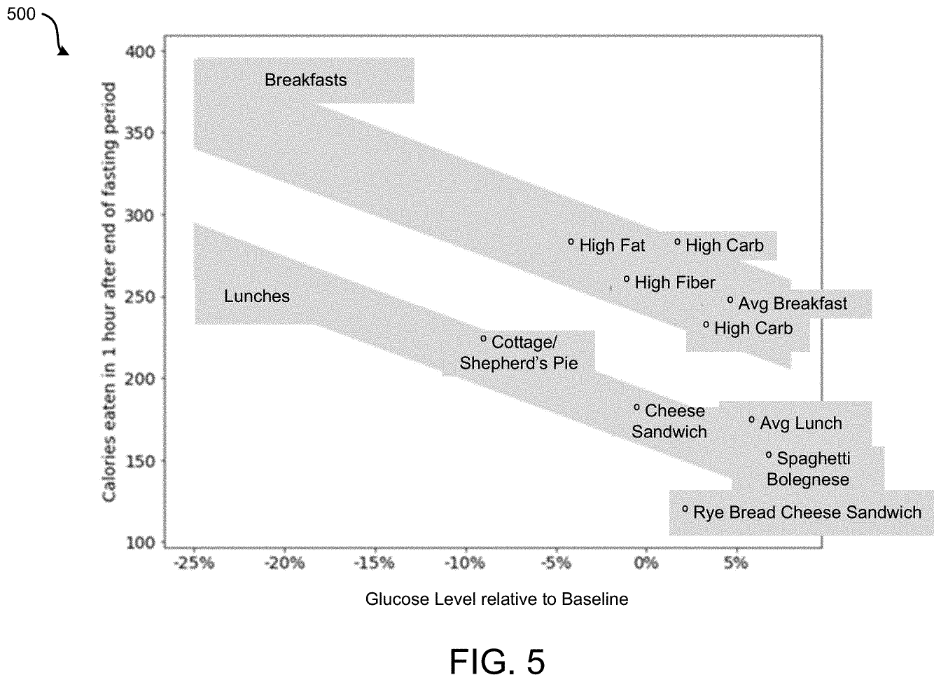

FIG. 5 shows a graph that illustrates differences in calories consumed after eating different meals. As illustrated, graph 500 shows that eating different foods evokes different glucose drops in the period after two hours than was expected from prior research. In this example, looking at meals with similar levels of carbohydrate but different composition, the behavior of glucose in the first two hours is quite similar between the meals, but the behavior in the period after the first two hours varies substantially based on the meal eaten, and the individual eating the meal.

Referring to graph 500, meals that elicit the largest glucose drops (such as Cottage/Shepherd's Pie) tend to be associated with the largest subsequent calorie intake. For the meals illustrated, graph 500 shows that the individuals with the largest glucose lows tend to eat on average more calories in hours 3-4 than the participants with the lowest glucose drops.

In some examples, the data used to generate graph 500 may include data obtained from both a clinical setting (e.g., clinic, hospital, lab, . . . ) and non-clinical settings. For instance, postprandial glucose and insulin responses may be obtained using a mixed-nutrient dietary challenge (e.g., 50 g fat, 85 g carbohydrate) in a clinical setting, and an at-home setting. The individuals may consume a range of standardized meals of differing macronutrient proportions alongside logging their remaining meals using a mobile application. It has been found that across thousands of standardized breakfasts, 25% of individuals that experience the largest dips in glucose in hours 2-3 after a meal reported greater hunger, choose to eat earlier (e.g., about 20 minutes sooner) and consume larger meals (+57 kcal), and more calories over the course of the full day.

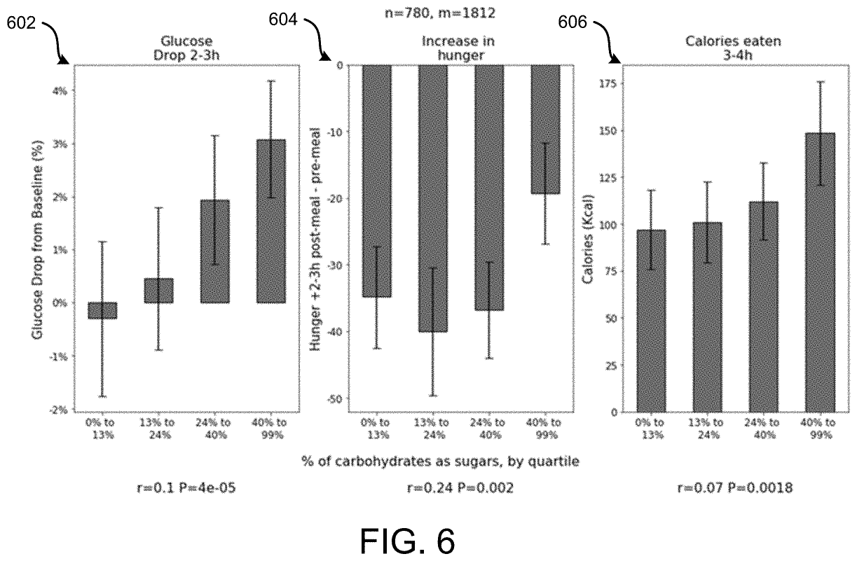

FIG. 6 shows increased hunger and calorie consumption based on glucose drops. As illustrated, the data illustrated within the glucose drop graph 602, the hunger graph 604, and the calories graph 606 was obtained from 1812 free-living meals consumed by 780 users. The meals eaten were between 400 and 600 calories, carbohydrates contributed 40-60% of total calories, and the meals were not preceded by a meal in the previous 3 hours. As shown, the meals were classified into quartiles according to the percentage of calories from sugar, as a proportion of the total carbohydrate, and compared to the glucose drop 2-3 h as illustrated by glucose drop graph 602, the increase in hunger after the meal in the hunger graph 604, and the calories eaten 3-4 h after the meal as illustrated in the calories eaten graph 606.

The meals with the highest proportion of carbohydrate in the form of sugars (40% to 99%) produced the biggest postprandial glucose dips (+3%). Compared to meals with the lowest proportion of sugars (0% to 13%), the higher sugar meals show an increase in hunger of the users as illustrated by hunger graph 604 and eating more calories in hours 2-3 (+51 kcal) as illustrated by calories graph 606. In some examples, hunger data is collected via a UI provided by an application that asks individuals to rate their hunger levels on a scale from 1-100, at intervals before and after the consumption of meals.

In some configurations, the nutritional service 120 weights the hunger score of a predicted glucose drop more heavily as compared to the glucose rise, and/or other responses. According to some examples, the timing of the glucose drop may indicate an insulin sensitivity of individuals. For example, insulin sensitive individuals may experience glucose drops soon (120 mins) after some insulinogenic meals but may rapidly return to baseline levels. Late glucose dips (240 minutes) may be seen in the less insulin sensitive individuals. The timing of the glucose dip may be strongly correlated with metabolic syndrome parameters with late glucose dips (at 4 hours) correlating with higher BMI, waist-hip ratio, insulin and HbalC.

In some configurations, the nutritional service 120 can adjust macronutrient content to reduce the predicted hunger of an individual. For example, the nutritional service 120 may adjust the meal size (in grams of carbohydrate), and the percentage of those carbohydrates that come from sugars to affect a glucose drop of an individual. The nutritional service 120 may also provide other personalized health scores 108A such as but not limited to metabolic risk, carbohydrate response, blood sugar control, lipid response, microbiome health, and the like. According to some examples, the user's personalized health scores are calculated by using the user's measured biological data compared with data from a large set of others so that the nutritional service 120 may determine where to place the user's score on a scale.

By sending these personalized health scores to a UI display service 118, the scores can be displayed directly to the individual who can therefore understand better their responses to food and why the nutritional service 120 is making the recommendations that it does.

Generating personalized weighting/scores for different biomarkers in a scientifically informed manner can be challenging, given limited data on how to compare risks associated with different biomarker responses or different foods. According to some configurations, expert dietary input is combined with biomarker predictions to help define the personalized weighting/scores service, 114. In some examples, dietary recommendations are obtained from experts in the field for a range of users with a state of health recognizable to the experts. For example: person A may be high BMI, prediabetic, age 55-65 etc., whereas person B may be aged 30-45, frequent jogger, eats largely a plant-based diet. These recommendations could be of various forms such as “Person A should be strongly discouraged from eating processed meat”, or “For Person B, the scores for low Glycemic Index carbs should be higher than those for good quality fats”. After obtaining the recommendations from the experts, the biomarker prediction service generates biomarkers for the participants/foods for which we have statements and machine learning may then be used to generate the personalized weighting/scores for each biomarker 106 and food data 113 that maximally satisfy the statements of the experts. Through this approach the hunger prediction/food scoring service 108 can generate food scores for any food and any individual, where the guidance can be personalized both to the individual's different biomarker responses and to the individual's health risks, and allows the combination of different biomarkers into a single score, solving for the problem of dealing with comparing foods where some biomarkers improve and some get worse.

According to some configurations, a prediction of a postprandial glucose dip may indicate that a user will experience increased hunger, and as a result consume more calories. These responses can be in part predicted based on both the amount of sugar in the meal and personal factors such as insulin sensitivity. In some examples, the glycemic dips are used by the nutritional service to predict both perceived hunger and the number of calories consumed at some point in time (e.g., 3 or more hours postprandially). This is linked to a person's insulin sensitivity and the sugar content, but not necessarily the glycemic index, of the meal.

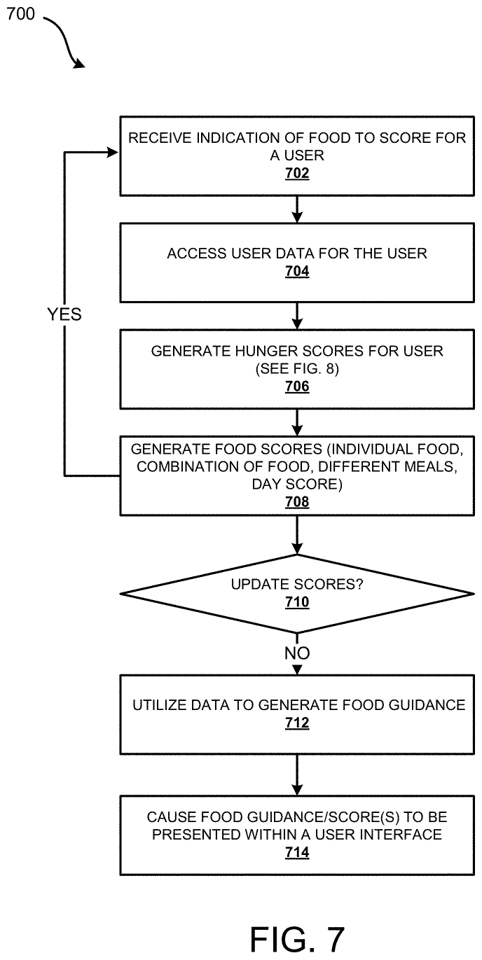

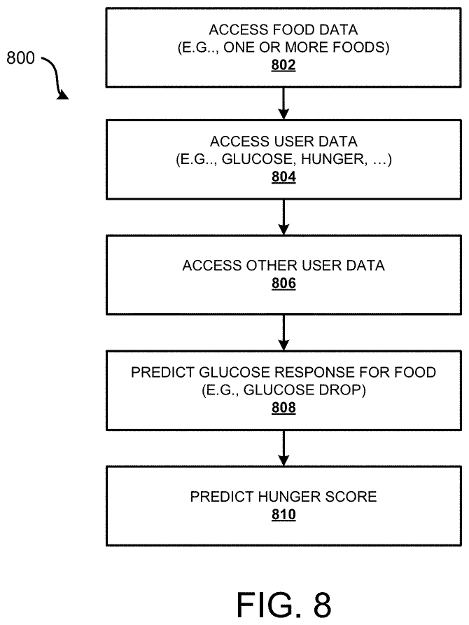

FIGS. 7 and 8 are flow diagrams showing routines 700, 800, in accordance with examples described herein. It should be appreciated that at least some of the logical operations described herein with respect to FIGS. 7 and 8, and the other FIGS., may be implemented (1) as a sequence of computer implemented acts or program modules running on a computing system and/or (2) as interconnected machine logic circuits or circuit modules within the computing system.

The implementation of the various components described herein is a matter of choice dependent on the performance and other requirements of the computing system. Accordingly, the logical operations described herein are referred to variously as operations, structural devices, acts, or modules. These operations, structural devices, acts, and modules may be implemented in software, in firmware, in special purpose digital logic and any combination thereof. It should also be appreciated that more or fewer operations may be performed than shown in the FIGS. and described herein. These operations may also be performed in parallel, or in a different order than those described herein.

FIG. 7 is a flow diagram showing routine 700 illustrating aspects of a mechanism disclosed herein for generating food guidance that is personalized for an individual using predicted hunger and food responses.

The routine 700 may begin at 702, where an indication is received for food to score for a user. As discussed above, the food can be a series of standardized meals, a single meal, a single food, or some other selection of food. In some examples, the user may provide an indication of food that has been eaten using a UI 134, or through some other mechanism. In some examples, the nutritional service 120 may provide one or more foods to score for a particular user. For instance, the nutritional service 120 may generate a list of foods based on preferences from a user, dietary restrictions, and the like.

At 704, user data for the user is accessed. As discussed above, the food guidance and scores that are generated are personalized for the user. In some configurations, the nutritional service 120 accesses data store 144 to access user data 140A. This data may be used by the nutritional service 120 to generate predictions, scores, and food guidance that is personalized for the user.

At 706, hunger scores are generated for the user. As discussed above, the nutritional service 120 may access one or more services to generate a hunger score in response to a single food, a combination of food, as well as a combination with foods that have already been eaten by the user. See FIG. 8 for more details and generating hunger scores.

At 708, food scores are generated for the user. As discussed above, the nutritional service 120 may access one or more services to generate predictions for a response to a single food, a combination of food, as well as a combination with foods that have already been eaten by the user.

At 710, a determination is made as to whether or not to update the hunger scores and/or the food scores. As discussed above, the nutritional service 120 may update one or more scores throughout a time period. For example, when a score has been calculated and more food has been eaten/scored then the nutritional service 120 may update the score to reflect the additional food. When the scores are to be updated, the routine returns to 702. When the scores are not to be updated, the routine flows to 712.

At 712, food guidance may be generated. As discussed above, the scores that are personalized for the user and other data (e.g., user data) may be used by the nutritional service 120 to generate food guidance as illustrated above to assist the user.

At 714, the food guidance/score(s) are presented within a UI. As discussed above, the nutritional service 120 may generate UI elements to display within UI 134 such that the user may readily determine the quality/health of the food they have eaten or may eat. In some examples, the scores may indicate how hungry one or more foods may make the individual feel, whether the food is healthy, or unhealthy and the like.

FIG. 8 is a flow diagram showing routine 800 illustrating aspects of a mechanism disclosed herein for generating hunger scores that are personalized for a user.

The routine 800 may begin at 802, where food data for a single food or combination of foods is accessed. As discussed above, the food data may include data such as nutritional content of the food, whether the food is processed, a level of food processing if the food is processed, and the like.

At 804, user data is accessed. As discussed above, the user data includes data associated with the individual to which the hunger score is associated. In some examples, the user data may include but is not limited to glucose response data, hunger data, biome data, and the like.

At 806, other user data may be accessed. As discussed above, the other user data includes data associated with other individuals and may include but is not limited to glucose response data, hunger data, biome data, and the like.

At 808, the glucose response for the food is predicted. As discussed above, the nutritional service 120 may predict the glucose drop for the food. In some examples, the nutritional service 120 may use one or more machine learning mechanisms to generate the glucose response and predict a time when the glucose drop is the largest. The machine learning mechanisms may be trained using data obtained from experts as well as other data. In some configurations, the machine learning mechanism is trained using thousands of samples from other users. In some examples, one or more machine learning mechanisms may be used to adjust scores for a particular user.

At 810, the hunger score is predicted. As discussed above, the nutritional service 120 may predict the hunger score for the food(s) based on the predicted glucose drop and/or other data. In some examples, the larger the predicted glucose drop the higher the score. The hunger score may be used by the nutritional service to adjust the macronutrients of the recommended foods.

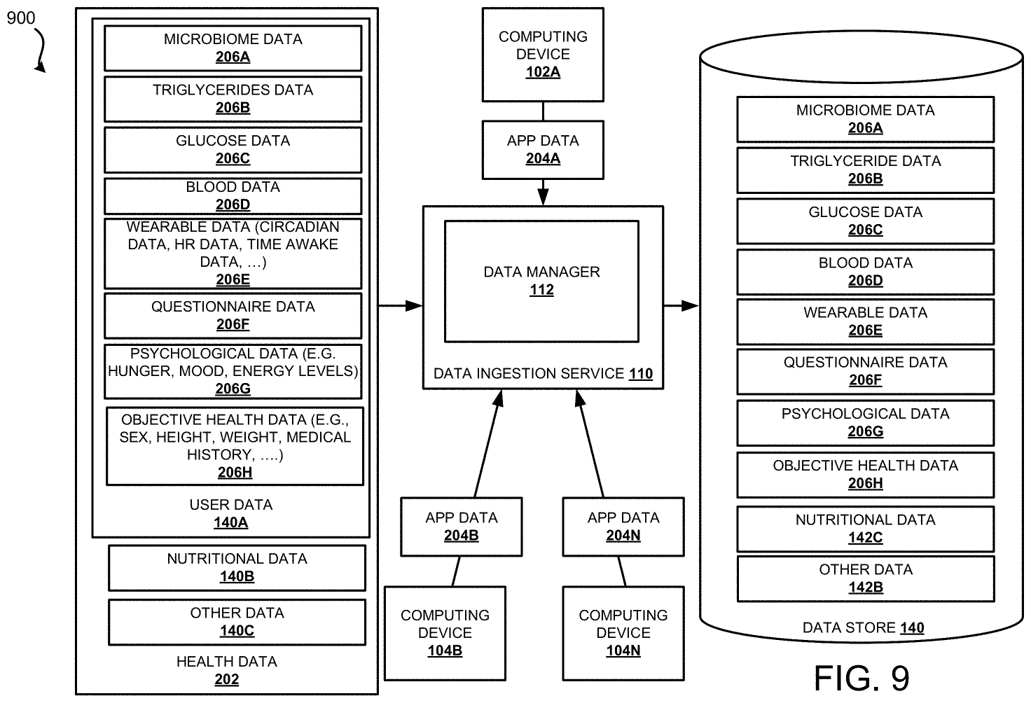

FIG. 9 is a block diagram depicting an illustrative operating environment 900 in which a data ingestion service 110 receives and processes data associated with data associated with at home measurements relating to intake and/or responses to food.

In some configurations, the data manager 112 is configured to receive data such as, health data 202 that can include, but is not limited to microbiome data 206A, triglycerides data 206B, glucose data 206C, blood data 206D, wearable data 206E that can include circadian data, questionnaire data 206F, psychological data (e.g., hunger, sleep quality, mood, . . . ) 206G, objective health data (e.g., height, weight, medical history, . . . ) 206H, nutritional data 140B, and other data 140C.

According to some examples, the microbiome data 206A includes data about the gut microbiome of an individual. The gut microbiome can host a large number of microbial species (e.g., >1000) that together have millions of genes.

The triglycerides data 206B may include data about triglycerides for an individual. In some examples, the triglycerides data 206B can be determined from an At Home Blood Test which in some cases is a finger prick on to a dried blood spot card. The glucose data 206C includes data about blood glucose. The glucose data 206C may be determined from various testing mechanisms, including at home measurements, such as a continuous glucose meter.

The blood data 206D may include blood tests relating to a variety of different biomarkers. As discussed above, at least some blood tests can be performed at home. In some configurations, the blood data 206D is associated with measuring blood sugar, insulin, c-peptides, triglycerides, cholesterol, IL-6 inflammation, ketone bodies, nutrient levels, allergy sensitivities, iron levels, blood count levels, HbAIc, and the like.

The wearable data 206E can include any data received from a computing device associated with an individual. For instance, an individual may wear an electronic data collection device 103, such as an activity-monitoring device, that monitors motion, heart rate, heart rate variability, determines how much an individual has slept that may include the types of sleep, the times an individual is awake, the number of calories burned, activities performed, blood pressure, body temperature, and the like. The individual may also wear a continuous glucose meter that monitors blood glucose levels.

The questionnaire data 206F can include data received from one or more questionnaires, and/or surveys received from one or more individuals. The psychological data 206G, that may be subjectively obtained, may include data received from the individual and/or a computing device that generates data or input based on a subjective determination (e.g., the individual states that they are still hungry after a meal, or a device estimates sleep quality based on the movement of the user at night perhaps combined with heart rate data). The objective health data 206H includes data that can be objectively measured, such as but not limited to height, weight, medical history, and the like.

The nutritional data 140B can include data about food, which is referred to herein as “food data”. For example, the nutritional data can include nutritional information about different food(s) such as their macronutrients and micronutrients or the bioavailability of its nutrients under different conditions (raw vs cooked, or whole vs ground up).

The other data 142B can include other data associated with the individual. For example, the other data 142B can include data that can be received directly from a computer application that logs information for an individual (e.g., food eaten, sleep, . . . ) and/or from the user via a user interface.

In some examples, different computing devices 102 associated with different users provide application data 204 to the data manager 112 for ingestion by the data ingestion service 110. As illustrated, computing device 102A provides app data 204A to the data manager 112, computing device 104B provides app data 204B to the data manager 112, and computing device 104N provides app data 204N to the data manager 112. There may be any number of computing devices utilized.

As discussed briefly above, the data manager 112 receives data from different data sources, processes the data when needed (e.g., cleans up the data for storage in a uniform manner), and stores the data within one or more data stores, such as the data store 144.

The data manager 112 can be configured to perform processing on the data before storing the data in the data store 144. For example, the data manager 112 may receive data for ketone bodies and then use that data to generate ketone body ratios. Similarly, the data manager 112 may process food eaten and generate meal calories, number of carbohydrates, fat to carbohydrate ratios, how much fiber consumed during a time period, and the like. The data stored in the data store 144, or some other location, can be utilized by the data service 120 to determine an accuracy of at home measurements of nutritional responses performed by users. The data outputted by the data service 108B to the nutritional service may therefore contain different values than are stored in the data store 144, for example if a food quantity is adjusted.

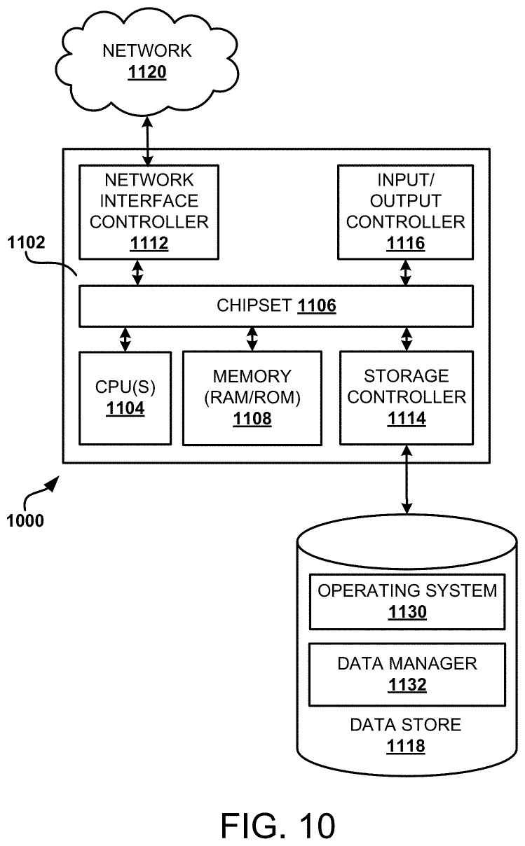

FIG. 10 shows an example computer architecture for a computer 1000. The computer architecture shown in FIG. 10 illustrates a conventional server computer, workstation, desktop computer, laptop, mobile device, tablet, network appliance, digital cellular phone, smart watch, or other computing device, and may be utilized to execute any of the software components presented herein. For example, the computer architecture shown in FIG. 10 may be utilized to execute software components for performing operations as described above. The computer architecture shown in FIG. 10 might also be utilized to implement a computing device 132, or any other of the computing systems described herein.

The computer 1000 includes a baseboard 1102, or “motherboard,” to which a multitude of components or devices may be connected by way of a system bus or other electrical communication paths. In one illustrative example, one or more central processing units (“CPUs”) 1104 operate in conjunction with a chipset 1106. The CPUs 1104 may be standard programmable processors that perform arithmetic and logical operations necessary for the operation of the computer 1000.

The chipset 1106 provides an interface between the CPUs 1104 and the remainder of the components and devices on the baseboard 1102. The chipset 1106 may provide an interface to memory 1106, such as RAM and/or read-only memory (“ROM”). The memory may store software components utilized for the operation of the computer 1000 in accordance with the examples described herein.

The computer 1000 may operate in a networked environment using logical connections to remote computing devices and computer systems through a network, such as the network 1120. The chipset 1106 may include functionality for providing network connectivity through a network interface controller, such as a cellular network adapter, WiFi network adapter, Ethernet adapter, and the like. The NIC 1112 is capable of connecting the computer 1000 to other computing devices over the network 1120.

The computer 1000 may be connected to a mass storage device 1116 that provides storage for the computer. The mass storage device 1116 may store system programs, application programs, other program modules and data, which have been described in greater detail herein. The mass storage device 1116 may be connected to the computer 1000 through a storage controller 1114 connected to the chipset 1106. The mass storage device 1116 may consist of one or more physical storage units. The storage controller 1114 may interface with the physical storage units through various type of interfaces.

The mass storage device 1116 described above, the computer 1000 may have access to other computer-readable storage media to store and retrieve information, such as program modules, data structures, or other data. By way of example, and not limitation, computer-readable storage media may include volatile and non-volatile, removable and non-removable media implemented in any method or technology.

The mass storage device 1116 may store an operating system 1130 utilized to control the operation of the computer 1000. According to some configurations, the operating system may include, but is not limited to the UNIX operating system, the LINUX operating system, the WINDOWS® operating system from MICROSOFT Corporation, the iOS operating system from APPLE Corporation, the ANDROID operating system from GOOGLE, and the like. Other operating systems may also be utilized. The mass storage device 1116 may store other system or application programs and data utilized by the computer 1000, such as components that include the data manager 1132, and/or any of the other software components and data described above. The mass storage device 1116 might also store other programs and data not specifically identified herein.

In one example, the mass storage device 1116 or other computer-readable storage media is encoded with computer-executable instructions that, when loaded into the computer 1000, implement the examples described herein. The computer 1000 has access to computer-readable storage media storing computer-executable instructions which, when executed by the computer 1000, may be configured to perform the various routines described above. The computer 1000 might also include computer-readable storage media for performing any of the other computer-implemented operations described herein.

The computer 1000 may also include one or more input/output controllers 1116 for receiving and processing input from a number of input devices, such as an electronic data collection device, a keyboard, a mouse, a touchpad, a touch screen, an electronic stylus, or other type of input device. Similarly, the input/output controller 516 may provide output to one or more types of output devices (e.g., a display, a projector, a printer, . . . ). The computer 1000 may not include all of the components shown in FIG. 10, may include other components that are not explicitly shown in FIG. 10, or may utilize an architecture completely different than that shown in FIG. 10.

Based on the foregoing, it should be appreciated that technologies for generating food guidance using predicted food responses have been presented herein. Moreover, although some of the subject matter presented herein has been described in language specific to computer structural features, methodological acts and computer readable media, it is to be understood that the invention defined in the appended claims is not necessarily limited to the specific features, acts, or media described herein. Rather, the specific features, acts and media are disclosed as example forms of implementing at least some of the claims.

The subject matter described above is provided by way of illustration only and should not be construed as limiting. Furthermore, the claimed subject matter is not limited to implementations that solve any or all disadvantages noted in any part of this disclosure. Various modifications and changes may be made to the subject matter described herein without following the examples and applications illustrated and described, and without departing from the true spirit and scope of the present invention, which is set forth in the following claims.

What is claimed is:

1. A method, comprising:
accessing glucose response data associated with glucose responses to food by users;
generating a hunger score, based at least in part on the glucose response data, wherein the hunger score is personalized for a user;
generating food guidance that is based, at least in part, on the hunger score; and
causing the food guidance to be presented within a user interface to the user.

2. The method of claim 1, further comprising:
accessing user data for the user, wherein the user data includes one or more of hunger data that indicates a hunger level of the user at a point in time after consuming one or more foods; and
wherein generating the hunger score is further based at least in part on the user data.

3. The method of claim 2, wherein the user data includes second glucose response data that indicates a glucose response of the user after consuming the one or more foods.

4. The method of claim 2, wherein the user data includes alertness data that indicates an alertness level of the user at a point in time after consuming one or more foods.

5. The method of claim 1, further comprising:
generating a plurality of food scores associated with one or more of different foods and a combination of foods; and wherein
generating the food guidance is further based, at least in part, on at least one of the food scores.

6. The method of claim 1, further comprising:
generating predicted glucose drops of the user based, at least in part, on a predicted consumption of foods by the user; and
wherein generating the hunger score, is further based at least in part on one or more of the predicted glucose drops.

7. The method of claim 1, further comprising selecting a glucose drop based, at least in part, on a size of the predicted glucose drop.

8. A system, comprising:
one or more processors, configured to perform acts, including
accessing glucose response data associated with glucose responses to food by users;
generating a hunger score, based at least in part on the glucose response data, wherein the hunger score is personalized for a user;
generating food guidance that is based, at least in part, on the hunger score; and
causing the food guidance to be presented within a user interface to the user.

9. The system of claim 8, the acts further comprising:
accessing user data for the user, wherein the user data includes hunger data that indicates a hunger level of the user at a point in time after consuming one or more foods; and
wherein generating the hunger score is further based at least in part on the user data.

10. The system of claim 9, wherein the user data includes second glucose response data that indicates a glucose response of the user after consuming the one or more foods.

11. The system of claim 9, wherein the user data includes alertness data that indicates an alertness level of the user at a point in time after consuming one or more foods.

12. The system of claim 8, the acts further comprising:
generating a plurality of food scores associated with one or more of different foods and a combination of foods; and wherein
generating the food guidance is further based, at least in part, on at least one of the food scores.

13. The system of claim 8, the acts further comprising:
generating predicted glucose drops of the user based, at least in part, on a predicted consumption of foods by the user; and
wherein generating the hunger score, is further based at least in part on one or more of the predicted glucose drops.

14. The system of claim 8, the acts further comprising selecting a glucose drop based, at least in part, on a size of the predicted glucose drop.

15. A non-transitory computer-readable storage medium having computer-executable instructions stored thereupon which, when executed by a computer, cause the computer to:
access glucose response data associated with glucose responses to food by users;
generate a hunger score, based at least in part on the glucose response data, wherein the hunger score is personalized for a user;
generate food guidance that is based, at least in part, on the hunger score; and
cause the food guidance to be presented within a user interface to the user.

16. The non-transitory computer-readable storage medium of claim 15, the computer-executable instructions when executed by the computer further causing the computer to:
access user data for the user, wherein the user data includes hunger data that indicates a hunger level of the user at a point in time after consuming one or more foods; and
wherein generating the hunger score is further based at least in part on the user data.

17. The non-transitory computer-readable storage medium of claim 16, wherein the user data includes one or more of second glucose response data that indicates a glucose response of the user after consuming the one or more foods and alertness data that indicates an alertness level of the user at a point in time after consuming one or more foods.

18. The non-transitory computer-readable storage medium of claim 15, the computer-executable instructions when executed by the computer further causing the computer to:
generate a plurality of food scores associated with one or more of different foods and a combination of foods; and
wherein generating the food guidance is further based, at least in part, on at least one of the food scores.

19. The non-transitory computer-readable storage medium of claim 15, the computer-executable instructions when executed by the computer further causing the computer to:
generate predicted glucose drops of the user based, at least in part, on a predicted consumption of foods by the user; and
wherein generating the hunger score, is further based at least in part on one or more of the predicted glucose drops.

20. The non-transitory computer-readable storage medium of claim 15, the computer-executable instructions when executed by the computer further causing the computer to select a glucose drop based, at least in part, on a size of the predicted glucose drop.
